(19) United States
(12) Patent Application Publication
(10) Pub. No.: US 2005/0015076 A1
(43) Pub. Date: Jan. 20, 2005

(54) Catheter application system

(75) Inventors: Carsten Giebmeyer, Witten (DE); Ross Sasser, Cumming, GA (US)

Correspondence Address: HOWSON AND HOWSON;ONE SPRING HOUSE CORPORATION CENTER, BOX 457, 321 NORRISTOWN ROAD, SPRING HOUSE, PA, 19477, US

(73) Assignee: Inmed Corporation, Duluth, GA (US)

(21) Appl. No.: 10/877,494

(22) Filed: Jun. 25, 2004

(30) Foreign Application Priority Data
Jun. 27, 2003 (DE) 103 29 126.1

(51) Int. Cl. A61M 27/00

(52) U.S. Cl. 604/544

(57) ABSTRACT

A urinary catheter application system utilizes a split insertion tip and stop, which can be removed from the catheter while the tip of the catheter is in the patient's bladder. The stop assembly includes a lubricant reservoir, and an opening permitting manual access to the catheter.

### FIELD OF THE INVENTION

This invention relates to a catheter application system, and in particular to an application system for introducing a urinary catheter into the urethra. The catheter system has an insertion sleeve to facilitate introduction of the catheter without importing microorganisms from the anterior section of the urethra into the bladder, so that the catheter remains as sterile as possible when introduced.

### BACKGROUND OF THE INVENTION

Catheters are used for diagnosis and treatment purposes, e.g., for suprapubic bladder drainage or urine elimination via the urethra. They are ordinarily supplied by the manufacturer in sterile packages. Prior to the application of a catheter, the area around the bodily orifice into which the catheter is to be introduced is washed and disinfected. However, absolute, 100%, disinfection is practically impossible to achieve, in particular with respect to the urethral orifice and anterior section of the urethra. Microbial contamination always lingers, especially in the anterior section of the urethra adjacent to the orifice, so that the sterile catheter must traverse a non-sterile area when introduced into the urethra. Microorganisms must be prevented from spreading from this urethral area into the bladder or posterior urethral areas to the greatest extent possible. Special precautions must therefore be taken during introduction, particularly in the case of long-term, or “indwelling” catheters, which may remain in place for several days.

It is known that sliding friction can be reduced during catheterization by wetting the outer surface of the catheter with a liquid, e.g., a lubricant or aqueous saline solution, so that the catheter can be introduced into a hollow organ of the human body causing as little pain as possible and without triggering further irritations. This can also be accomplished with other coatings to improve sliding characteristics. A hydrophilic outer surface over the length of the catheter shaft is also frequently used for this purpose, and is activated by means of an aqueous liquid prior to catheter application. Water molecules bind to the hydrophilic surface when the surface is wetted with water. This yields a soft, smooth surface that enables comfortable introduction of the catheter. These designs have in the past been proven effective in preventing discomfort during the introduction of a catheter.

It is also known to provide both the inner surface and the outer surface of the catheter with an anti-microbial coating, e.g., a coating of silver, to reduce bacterial adherence.

German patent application DT 2456980 A1, and U.S. Pat. Nos. 3,421,509 and 4,652,259, disclose urethral catheter application systems with an insertion sleeve which is introduced into the anterior area of the urethra infected with bacteria until a stop, e.g., a ring, restricts the introduction of the insertion sleeve into the urethra. After the insertion sleeve has been introduced into the urethra, a catheter can be pushed through the insertion sleeve. The catheter opens a slit at the distal end of the insertion sleeve, and can then be advanced into the bladder. The insertion creates a sterile passage, allowing the catheter to be introduced through the microbe-contaminated area of the urethra without transporting large quantities microorganisms toward the bladder.

Catheter application systems in the prior art provide insertion aids that completely surround the catheter shaft, and consequently remain on the catheter after it has been introduced.

An object of the invention is to provide a catheter application system that avoids the disadvantages of prior art; which is particularly easy, quick and inexpensive to use; and which helps to avoid or reduce catheter-associated infections, in particular, urethral infections. It is also an object of the invention to provide a catheter application system in which the insertion sleeve and stop can be easily and readily removed, so that the application system is particularly advantages for use with indwelling catheters.

### SUMMARY OF THE INVENTION

The urinary catheter application system according to the invention comprises a catheter adapted for introduction through the urethra into the bladder for bladder drainage, a sheath surrounding the catheter and having a distal end, and an insertion aid located at the distal end of the sheath, through which the catheter is movable. The insertion aid comprises, as a first component, an insertion sleeve for introduction into the urethra, the insertion sleeve having a passage through which the catheter can slide, and, as a second component, a stop for restricting the introduction of the insertion sleeve into the urethra. At least one of these first and second components is composed of two separable parts allowing its removal from the catheter while the catheter extends into the urethra. Preferably, the two separable parts, when joined together, surround the catheter when the catheter extends through the insertion sleeve.

In a preferred embodiment, the insertion sleeve has a normally closed seal, openable by movement of the catheter through the passage of the insertion sleeve. This seal is operative even where the insertion sleeve is composed of two separable parts. In the preferred embodiment, the insertion aid also includes a liquid reservoir, filled with a liquid from the group consisting of lubricant gel, water, and physiological saline solution, that can be penetrated by the catheter during movement of the catheter through said insertion aid.

The invention also resides in a novel process for the application of a urinary catheter to a patient. The catheter application process comprises the steps of passing a catheter into the patient's bladder through the urethra by inserting the catheter through an insertion aid comprising as a first component, an insertion sleeve for introduction into the urethra, said insertion sleeve having a passage through which the catheter can slide, and, as a second component, a stop for restricting the introduction of the insertion sleeve into the urethra, and, while the catheter tip is in the patient's bladder, removing at least one, and preferably both, of the first and second components from the catheter. Where the removed component is composed of two separable parts that, when joined together, surround the catheter when the catheter extends through the insertion sleeve, the removal step is carried out by separating said two parts from each other.

The advantage to the split insertion aid design is that the insertion aid can be easily removed from the catheter without passing the entire catheter through the insertion aid. This would not be possible in the case of a urine bag rigidly attached to the catheter. As a result, the microbe-contaminated insertion aid, or at least the contaminated insertion sleeve, can be easily removed from the catheter following its application, making it possible to avoid an ascending infection.

After preparing the catheter application system according to the invention, the urinary catheter is applied by introducing the insertion sleeve, which is supported from inside by the catheter tip, into the anterior area of the urethra of the patient adjacent the orifice until the stop element prevents further insertion. The catheter is then passed through the insertion aid, catheter tip first. As the catheter tip passes through the insertion aid, it opens the sealing element. The catheter is then advanced through the insertion aid until its tip reaches the bladder of the patient. If a balloon is provided adjacent the catheter tip for catheter retention, the balloon is expanded. Thereafter, the insertion sleeve is pulled out of the urethra, and the insertion sleeve, and preferably the entire insertion aid, is disassembled by splitting it, so that it can be removed from the catheter.

In another embodiment, the sealing element of the catheter application system has a perforation or cross-shaped slit, so that opening becomes possible by penetrating the perforation or cross-shaped slit while passing through the catheter. The advantage to designing the sealing element with a perforation or cross-shaped slit is that no additional parts are required to ensure penetrability of the sealing element, and that the sealing element retains its primarily closed tip shape.

Each of the two components of the insertion aid, that is, the insertion sleeve and the stop, preferably consists of at least two segments that, when joined, form an annulus capable of enveloping the catheter. The segments hence each have an area adjacent to the through hole. In the simplest case, each of the splittable insertion aid components consists of two symmetrical segments. For example, if the stop element is designed as a ring, each of its two segments constitutes half a ring. The advantage to the segmented embodiment is that a simple partition of insertion aid components ensures splittability. As a result, this embodiment is simple and inexpensive to manufacture.

The splittable insertion aid component segments may be held together at predetermined breaking lines, by adhesive tape, crepe tape, and/or a band having hook and loop fasteners. Alternatively, the segments may be formed with interlocking elements, in the form of projections and projection-receiving holes. An embodiment with interlocking elements is particularly advantageous, since it obviates additional components to hold the segments of the splittable insertion aid together. No additional production costs are incurred. In addition, catheter application is no more difficult than in the case of known catheters having conventional insertion aids.

The insertion aid of the catheter application system, in particular the stop element, is preferably designed as a handle, wherein the handle preferably incorporates a through hole. The through hole can be present on one or both sides. To this end, the stop element can be composed of several assembled parts. A bilateral through hole in the handle can be used to grip the catheter in the through hole as it is passed through the insertion aid, e.g., with two fingers, thereby facilitating catheter application. In a unilateral through hole, the catheter inside the handle can be pressed against the inside of the handle, e.g., with a finger of the person applying the catheter, and thereby be held fast.

The insertion aid preferably has a removable protective cover over the insertion sleeve. This protects the insertion sleeve against mechanical damage and keeps the insertion sleeve sterile. The protective cover can also simultaneously cover the surface of the stop element.

The catheter preferably has a hydrophobic/oleophobic outer surface and/or an antimicrobial, preferably silver, coating on its outer and/or inner surfaces.

The protective cover and/or insertion sleeve are each preferably filled with a liquid. In addition, the insertion aid can incorporate a liquid reservoir filled with liquid that can be penetrated by the catheter during its application. These embodiments ensure that the catheter is wetted with liquid during introduction into a bodily orifice. This permits a largely pain-free insertion. The liquid reservoir guarantees that enough liquid is metered based on the personal needs of the user. Gentle catheterization without additional aids and catheter preparations is enabled.

The liquids preferably include a lubricant gel and/or water and/or physiological saline solution (0.9% w/w solution).

A pre-connected collection container, e.g., a urine bag, is preferably secured to the catheter of the catheter application system. During use of the catheter application system, this prevents the inner surface of the catheter from being exposed to microbial contamination when connecting the catheter with a collection container.

In an indwelling urinary catheter, the tip area of the catheter has an activatable retaining system, e.g., an inflatable balloon or cuff, which, when inflated, locks catheter tip in the bladder of the patient. A hollow space is provided around the catheter tip, which can be filled with a fluid through a tubular line that runs along the catheter. As a result, reliable placement of an indwelling catheter in the bladder can be ensured.

In particular for urinary catheters, the insertion sleeve has a sleeve length between 1.55 and 2 cm, and a diameter adjusted to the respective outer diameter of the used indwelling catheter. These dimensions correspond to those of the urethra, and in particular the length of the part of the urethra that is naturally contaminated with microbes.

Reverse sliding of the catheter back out of the urethra during its introduction can be permitted unintentionally, and can very easily result in contamination. A preferred locking device that allows the catheter to move through the insertion aid in only the insertion direction, may be provided inside the insertion aid of the catheter application system. The locking device prevents the catheter from sliding back out of the urethra.

Additional advantages may be gleaned from the specification and attached drawings. The previously mentioned, and yet to be specified, features of the invention can each be used individually or in combination. The cited embodiments are not to be construed as a comprehensive listing, but rather as being of an exemplary nature.

The invention will be explained in greater detail below based on exemplary embodiments with reference to the drawings.

### BRIEF DESCRIPTION OF THE DRAWINGS

FIG. 1 is a perspective view showing a catheter application system according to the invention;

FIG. 2a is an exploded view of the insertion aid;

FIG. 2b shows the insertion aid of FIG. 2a in its assembled condition; and

FIG. 3 is an exploded view of an embodiment of a stop element with a unilateral through hole.

The figures in the drawings provide a highly diagrammatic representation of the subject matter of the invention, and are not to scale. The individual components of the subject matter of the invention are shown in such a way as to effectively illustrate their structure.

### DETAILED DESCRIPTION OF THE PREFERRED EMBODIMENT

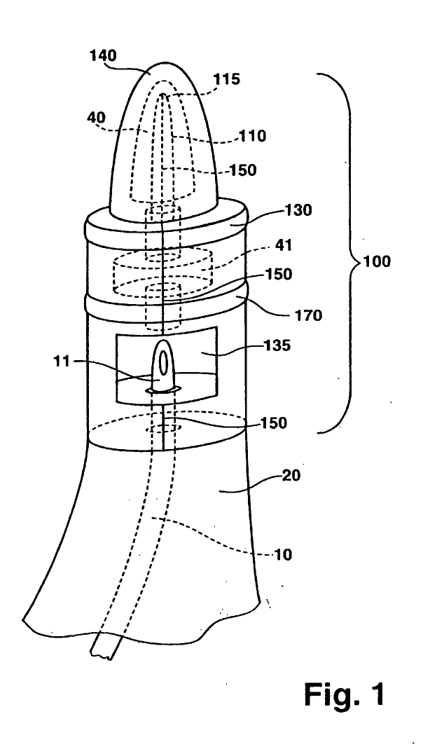

FIG. 1 shows the catheter application system according to the invention. The entire system consists of a closed, sterile catheter system comprising a catheter 10 made of silicone, PVC, polyurethane (PU), or latex, which enables sterile catheterization. The closed, sterile system, a silver coating and a hydrophobic/oleophobic coating on both the outer and inner surfaces of the catheter 10 contribute to the prevention of catheter-induced urinary tract infections. The coating is applied using nano-coating technology. The catheter 10 can be either an indwelling catheter designed for use over an extended time, or an intermittent catheter intended for insertion only for the time required for bladder drainage.

The catheter 10 has a rounded catheter tip 11 with at least one drainage hole, or “eye,” provided with rounded edges to improve the sliding characteristics of the catheter. The catheter 10 is initially disposed within a bag-like tubular sheath 20 composed of a plastic film, for example polyethylene film, through which the catheter can slide. A distal end of the sheath 20 is provided with an insertion aid, generally indicated by reference numeral 100, through which the catheter can be passed.

The sheath 20 is secured to the insertion aid, preferably using adhesive tape, heat-shrink tubing, or another form of fastener such as a band 170 having cooperating hook and loop fastener elements. The plastic bag envelops the catheter, keeping the catheter, and other components that are inside the bag, in a sterile condition.

The insertion aid 100 consists of assembled insertion aid components, namely an insertion sleeve 110 which includes a sealing element 115, and a stop element 130 to limit introduction of the insertion sleeve into a bodily orifice. The sealing element 115 can be opened for passage of the catheter through sleeve 110. In the preferred embodiment, the sealing element is opened as a result of penetration by the catheter tip 11. To this end, the sealing element 115 may be, for example, a normally closed slit or slit-like perforation in the insertion sleeve 110, or a cross-shaped slit. The insertion sleeve, is preferably composed of a soft plastic, and may be in the form of a membrane.

The stop element 130 extends outside the sheath 20. The insertion sleeve 110 and stop element 130 are splittable. That is, they can be divided into at least two segments, making them removable from the catheter while the catheter extends through the insertion aid. To this end, a predetermined break 150 is provided both in the stop element 130 and in the insertion sleeve 110. The dividable insertion sleeve 110 serves as an insertion aid for the catheter, bridging a short distal portion of the urethra, preferably over a distance of about 1.55 to 2.0 centimeters. The stop element 130 is preferably sufficiently long that it can be easily gripped with one hand and used for manipulation of the insertion aid. A liquid reservoir 41 and a through hole 135 are situated within the elongated stop element. When the catheter is passed through the stop element 130 and penetrates the liquid reservoir 41, it can be gripped by two fingers via the through hole 135.

The basic element of the insertion aid, i.e., the stop element, is made out of hard plastic. The retaining band 170, with cooperating hook-and-loop fasteners (crepe tape), holds the segments of the stop element 130 together, while simultaneously securely binding the sheath 20 to the stop element 130. As mentioned previously, this can also be accomplished with a piece of heat-shrink tubing or adhesive tape.

The insertion sleeve 110 is provided with a protective cover 140 that has an enlarged base, and hence also covers a part of the stop element. The hollow space 40 formed between the protective cover and the insertion sleeve 110 is filled with a liquid, e.g., a water-based lubricant gel. The insertion sleeve 110 is preferably also filled with lubricant gel in advance. Thus, sufficient lubricant gel is provided on the catheter and on the insertion sleeve. The protective cover 140 is also made out of hard plastic, and can be easily removed by simply pulling, or turning and pulling.

The end of the catheter remote from the catheter tip usually has a funnel (not shown). This funnel may be pre-connected to a urine drainage system such as a urine bag, by the manufacturer. The urine bag is preferably secured to the catheter by means of a piece of heat-shrink tubing. The sheath of the catheter application system ends at the catheter funnel.

The catheter application system can also have an anti-urine reflux system and/or a loop secured to or integral with the urine bag, and a hole for holding and hanging the bag. Indwelling urinary catheters usually also consist of a urine collecting site at the point where the urine bag is connected to the catheter, a Pasteur drip chamber for flow interruption, an anti-reflux valve, a vent with anti-microbial function and/or a drip-free outlet.

The catheter application system is provided in a sterile outer packaging. This outer packaging is configured in such a way that it can be peeled back from the insertion aid toward the urine bag for the purpose of catheter application.

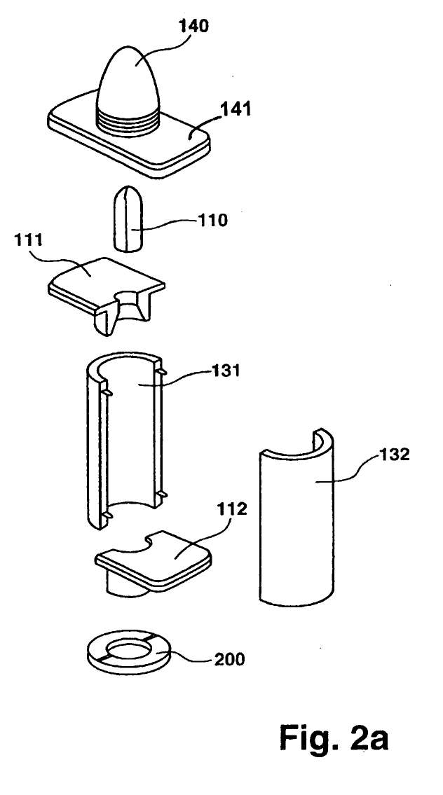

In FIGS. 2a and 2b, a protective cover, an insertion aid 110 and a splittable stop element are shown. The protective cover comprises a hollow, bullet-shaped, element 140 for receiving an insertion sleeve, and a generally rectangular element 141 having a recessed bottom (not shown) for accommodating wings on the stop element. The dimensions of the stop element are such that it can be used as a handle. The stop element consists of several parts, specifically two wing segments 111 and 112, two handle segments 131 and 132, and a sealing ring 200. The wing segments together yield a surface area forming the stop at the penis tip.

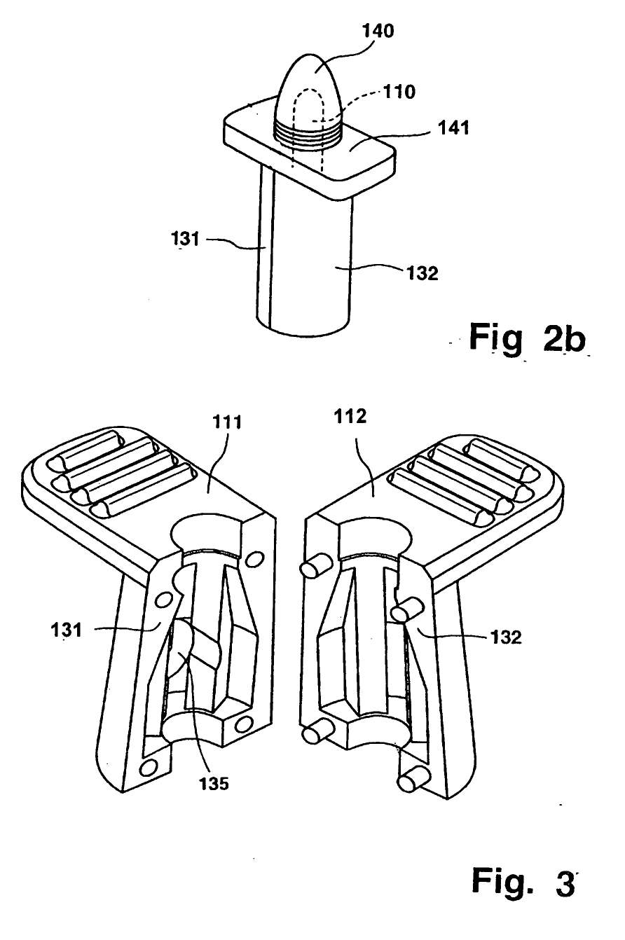

FIG. 3 shows an embodiment of a stop element with a unilateral through hole corresponding to hole 135 in FIG. 1. The stop element consists of two wing segments 111 and 112, and two handle segments 131 and 132. The stop element consists of two parts in all, which are shown split apart in the figure.

The surface of the stop element, which touches the body of the patient during introduction to limit the introduction of the insertion aid, is ribbed. The two parts are splittably connected, preferably by peg-shaped projections on one part that fit into matching holes on the opposite part. Of course, various alternative means of connection of the two parts can be used, such as interlocking ribs When assembled, the parts form an interior hollow space. This hollow space incorporates webs that precisely guide a catheter passing through the interior of the stop element. A first part consists of a handle segment 131 and a wing segment 111 comprising a single piece. This first part has a through hole 135. As shown, four projection-receiving holes are formed in this first part. The second part is also a single piece comprising a handle segment 132 and a wing segment 112. However, it has no through hole. The through hole is therefore unilateral. The four projections are formed on the second part.

In another embodiment, at least the handle segments 131 and 132 are made out of a soft, deformable material, preferably with resilient characteristics. Users can employ their fingers to deform such handle segments without exerting much force, to an extent where a catheter passed through the stop element can be held fixed in any position. In this embodiment (not shown) unintentional reverse slipping movement of the a catheter can be prevented by manual squeezing, and there is no need for a through hole, as shown in FIG. 1 and FIG. 3.

1. A urinary catheter application system comprising
a catheter adapted for introduction through the urethra into the bladder for bladder drainagec Nu said catheter having a tip;
a sheath surrounding the catheter and having a distal end; and
an insertion aid located at the distal end of the sheath, said catheter being movable through the insertion aid;
wherein the insertion aid comprises, as a first component, an insertion sleeve for introduction into the urethra, said insertion sleeve having a passage through which the catheter can slide, and, as a second component, a stop for restricting the introduction of the insertion sleeve into the urethra; and
wherein at least one of said components is composed of two separable parts allowing removal of said one of said components from the catheter while the catheter extends into the urethra.

2. A urinary catheter application system according to claim 1, in which the insertion sleeve is composed of two separable parts that, when joined together, surround the catheter when the catheter extends through the insertion sleeve.

3. A urinary catheter application system according to claim 2, in which the insertion sleeve has a normally closed seal, openable by movement of the catheter through said passage.

4. A urinary catheter application system according to claim 1, in which the stop is composed of two separable parts that, when joined together, surround the catheter when the catheter extends through the insertion sleeve.

5. A urinary catheter application system according to claim 1, in which the insertion sleeve is composed of two separable parts that, when joined together, surround the catheter when the catheter extends through the insertion sleeve, and in which the stop is also composed of two separable parts that, when joined together, surround the catheter when the catheter extends through the insertion sleeve.

6. A urinary catheter application system according to claim 1, in which the insertion aid includes a liquid reservoir filled with liquid that can be penetrated by the catheter during movement of the catheter through said insertion aid.

7. A urinary catheter application system according to claim 6, in which said liquid is from the group consisting of lubricant gel, water, and physiological saline solution.

8. A process for the application of a urinary catheter to a patient comprising the steps of:
passing a catheter having a catheter tip into the patient's bladder through the urethra by inserting the catheter through an insertion aid comprising as a first component, an insertion sleeve for introduction into the urethra, said insertion sleeve having a passage through which the catheter can slide, and, as a second component, a stop for restricting the introduction of the insertion sleeve into the urethra; and
while the catheter tip is in the patient's bladder, removing at least one of said components from the catheter.

9. A process according to claim 8, in which both of said components are removed while the catheter tip is in the patient's bladder.

10. A process according to claim 8, in which said at least one of said components is composed of two separable parts that, when joined together, surround the catheter when the catheter extends through the insertion sleeve, and in which the step of removing at least one of said components from the catheter is carried out by separating said two parts from each other.
